(19) United States
(12) Patent Application Publication
(10) Pub. No.: US 2021/0353782 A1
(43) Pub. Date: Nov. 18, 2021

(54) PHOTOACOUSTIC ION INDICATORS

(71) Applicants: Barbara SMITH, Tempe, AZ (US); Christopher MIRANDA, Mesa, AZ (US); Madeleine HOWELL, Cambridge, MA (US); Joel LUSK, Mesa, AZ (US)

(72) Inventors: Barbara SMITH, Tempe, AZ (US); Christopher MIRANDA, Mesa, AZ (US); Madeleine HOWELL, Cambridge, MA (US); Joel LUSK, Mesa, AZ (US)

(73) Assignee: Arizona Board of Regents on behalf of Arizona State University, Scottsdale, AZ (US)

(21) Appl. No.: 17/321,722

(22) Filed: May 17, 2021

Related U.S. Application Data
(60) Provisional application No. 63/025,803, filed on May 15, 2020.

(51) Int. Cl. A61K 49/22; A61B 5/00; G01N 33/50; G01N 33/84

(52) U.S. Cl. CPC A61K 49/22; A61B 5/0095; G01N 33/84; G01N 33/5005; A61B 5/0042

(57) ABSTRACT

A system for measuring the membrane potential of a neuron is disclosed. The system comprises one or more photoacoustic ion indicators, each comprising a metal chelating agent linked to a chromophore molecule. The metal chelating agent is configured to selectively bind to one of sodium ions, calcium ions, and potassium ions. The system further comprises a photoacoustic probe including a laser configured to emit a light signal to the chromophore and an ultrasound transducer configured to receive a photoacoustic signal in response to the light signal. The system further comprises a processor configured to receive the photoacoustic signal from the ultrasound transducer, determine a quantity of photoacoustic ion indicators exhibiting the shift, and calculate a membrane potential of the neuron based on quantity of photoacoustic ion indicators exhibiting the shift.

### CROSS-REFERENCE TO RELATED APPLICATIONS

This application claims the benefit of priority to U.S. Provisional Application No. 63/025,803 entitled “Photoacoustic Ion Indicators,” filed May 15, 2020, which is incorporated herein by reference in its entirety.

### TECHNICAL FIELD

The present disclosure relates generally to methods, systems, and apparatuses related to detecting and measuring the membrane potential of neurons. The disclosed techniques may be applied to various tissues as a research tool, for example, in vitro neural tissue and/or in vivo neural tissue (e.g., brain tissue).

### BACKGROUND

The activity of neurons has long been studied to characterize and understand neurological activity. Neurons are electrically excitable cells that serve as a primary component of the nervous system in most animals. Neurons are bundled together to form nerve tracts in the central nervous system and nerves in the peripheral nervous system, thus creating the basic pathway of neurological activity that facilitates communication between different parts of the body to convey information and coordinate actions.

Neurological activity is directly related to changes in membrane potential due to ion flux across the neuron's membrane. In the resting state, a surplus of positively charged ions outside of the cell create a negative concentration gradient across the membrane. In response to a stimulus, some sodium channels open allowing an influx of sodium ions (Na⁺) that results in depolarization of the cell. A threshold amount of depolarization triggers the opening of voltage-gated sodium channels that allow a massive rush of sodium ions into the cell, thereby causing an action potential to pass across the axon as the depolarization spreads. Thereafter, voltage-gated potassium channels open (while the voltage-gated sodium channels close) and cause an outflow of potassium ions (K⁺) to repolarize the cell, eventually returning it to the resting potential. Finally, an influx of calcium ions (Ca²⁺) through voltage-gated calcium channels triggers the release of neurotransmitters from the axon terminal to initiate synaptic transmission.

Due to this close relationship between concentration gradients and signal transmission across the neuron, direct imaging related to the concentration of these ions reveals the neurological activity and facilitates study of nervous system function and disorders thereof. Current approaches to imaging ion flux in neurons utilize fluorescent ion indicators (FIIs) comprising a metal chelating agent (i.e., a metal chelator) combined with a fluorescent contrast agent (i.e., a genetically encoded fluorophore, imaging dye, etc.). Together, the complex selectively binds a specific metal ion such as Na⁺ or Ca²⁺ and fluoresces and/or causes a shift in fluorescence in response to enable visualization of the concentration gradient. At present, FIIs are the most widely adopted tool for visualizing neuronal activity of a single cell (i.e., high resolution) through imaging techniques.

Although available FIIs have served as a gold standard for visualizing the flow of ions across the neuron membrane, they suffer from several drawbacks. Calcium indicators provide only an indirect measure of the action potential and are incapable of providing direct measurement due to the delay between the action potential and the calcium influx. On the other hand, while sodium indicators are able to more directly measure action potential, their development has proven very challenging and thus the available options are limited and generally offer poor contrast. Potential for development is limited by the fact that FIIs require a highly fluorescent molecule, which is a rarer trait that restricts the choice of materials. Even further, the reliance on fluorescence for visualization naturally limits the depth at which imaging can occur.

As such, it would be advantageous to have a method of imaging ion flux without reliance on fluorescent molecules in order to provide an abundant range of materials for development of ion indicators. It would be further advantageous to have an ion indicator configured for high resolution imaging at greater depths.

### SUMMARY

A photoacoustic ion indicator for detecting ion concentration within a neuron is provided. The ion indicator comprises a metal chelating agent comprising one or more polar groups, wherein the metal chelating agent is configured to selectively bind to an ion selected from the group consisting of sodium, calcium, and potassium; a chromophore linked to the metal chelating agent, wherein the chromophore molecule exhibits a shift of at least one light absorption characteristic upon binding of the metal chelating agent to the ion; and one or more acetoxymethyl esters bound to the one or more polar groups and configured to be cleaved from the one or more polar groups by an esterase within the neuron, wherein the photoacoustic ion indicator is permeable through a membrane of the neuron when the one or more acetoxymethyl esters are bound to one or more polar groups, and wherein the photoacoustic ion indicator is impermeable through the membrane of the neuron when the one or more acetoxymethyl esters are cleaved from the one or more polar groups.

According to some embodiments, the ion is a sodium ion, and the metal chelating agent comprises 15-crown-5 ether configured to selectively bind to the sodium ion.

According to some embodiments, the ion is a calcium ion, and the metal chelating agent comprises BAPTA motif configured to selectively bind to the calcium ion.

According to some embodiments, the ion is a potassium ion, and the metal chelating agent comprises 18-crown-6 ether configured to selectively bind to the potassium ion.

According to some embodiments, the photoacoustic ion indicator has a substantially neutral charge when the one or more acetoxymethyl esters are bound to one or more polar groups, and wherein the photoacoustic ion indicator has a substantially negative charge when the one or more acetoxymethyl esters are cleaved from the one or more polar groups.

According to some embodiments, a dissociation constant of the metal chelating agent binding the ion is less than about 50 mM.

According to some embodiments, the chromophore has an extinction coefficient greater than about 103 M-1 cm-1.

According to some embodiments, the chromophore comprises a linear acene.

According to some embodiments, the at least one light absorption characteristic comprises one or more of an absorption wavelength range, a peak absorption wavelength, a total absorption value, and an absorption coefficient. According to additional embodiments, the chromophore has a peak absorption wavelength greater than about 350 nm after the shift.

A system for measuring the membrane potential of a neuron is also provided. The system comprises one or more photoacoustic ion indicators, each photoacoustic ion indicator comprising: a metal chelating agent configured to selectively bind to an ion selected from the group consisting of sodium, calcium, and potassium; and a chromophore linked to the metal chelating agent, wherein the chromophore exhibits a shift of at least one light absorption characteristic upon binding of the metal chelating agent to the ion; a photoacoustic probe comprising: a laser configured to emit a light signal, wherein the chromophore is configured to absorb the light signal, and an ultrasound transducer configured to receive a photoacoustic signal from each photoacoustic ion indicator in response to the light signal; a processor; and a non-transitory, computer-readable medium storing instructions that, when executed, cause the processor to: receive the photoacoustic signals from the ultrasound transducer; determine, based on the photoacoustic signals, a quantity of the one or more photoacoustic ion indicators exhibiting the shift; and calculate a membrane potential of the neuron based on the quantity of the one or more ion indicators exhibiting the shift.

According to some embodiments, the ion is a sodium ion, and the metal chelating agent comprises 15-crown-5 ether configured to selectively bind to the sodium ion.

According to some embodiments, the ion is a calcium ion, and the metal chelating agent comprises BAPTA motif configured to selectively bind to the calcium ion.

According to some embodiments, the ion is a potassium ion, and the metal chelating agent comprises 18-crown-6 ether configured to selectively bind to the potassium ion.

According to some embodiments, the photoacoustic ion indicator is configured to be loaded into the neuron by whole-cell patch clamp electrophysiology.

According to some embodiments, the photoacoustic ion indicator is configured to be loaded into the neuron by passive cell loading,

According to some embodiments, the photoacoustic ion indicator further comprises one or more acetoxymethyl esters bound to one or more polar groups of the metal chelating agent and configured to be cleaved from the one or more polar groups by an esterase within the neuron, wherein the photoacoustic ion indicator is permeable through a membrane of the neuron when the one or more acetoxymethyl esters are bound to one or more polar groups, and wherein the photoacoustic ion indicator is impermeable through the membrane of the neuron when the one or more acetoxymethyl esters are cleaved from the one or more polar groups.

According to some embodiments, the photoacoustic ion indicator has a substantially neutral charge when the one or more acetoxymethyl esters are bound to one or more polar groups, and the photoacoustic ion indicator has a substantially negative charge when the one or more acetoxymethyl esters are cleaved from the one or more polar groups.

According to some embodiments, the at least one light absorption characteristic comprises one or more of an absorption wavelength range, a peak absorption wavelength, a total absorption value, and an absorption coefficient.

According to some embodiments, the chromophore has a first absorption coefficient when the metal chelating agent is unbound and a second absorption coefficient, different from the first absorption coefficient, when the metal chelating agent is bound to the ion, and the instructions that cause the processor to determine a quantity of the one or more ion indicators exhibiting the shift comprise instructions that, when executed, cause the processor to determine a quantity of the one or more photoacoustic ion indicators having the second absorption coefficient.

According to some embodiments, the chromophore has a first peak absorption wavelength when the metal chelating agent is unbound and a second peak absorption wavelength, different from the first peak absorption wavelength, when the metal chelating agent is bound to the ion, and the instructions that cause the processor to determine a quantity of the one or more ion indicators exhibiting the shift comprise instructions that, when executed, cause the processor to determine a quantity of the one or more photoacoustic ion indicators having the second peak absorption wavelength.

A method of measuring the membrane potential of a neuron is also provided. The method comprises loading one or more photoacoustic ion indicators into the neuron, wherein each photoacoustic ion indicator comprises: a metal chelating agent configured to selectively bind to an ion selected from the group consisting of sodium, calcium, and potassium, and a chromophore linked to the metal chelating agent, wherein the chromophore exhibits a shift of at least one light absorption characteristic upon binding of the metal chelating agent to the ion; emitting, by a light source, a light signal configured to be absorbed by the chromophore; receiving, by a photoacoustic probe, a photoacoustic signal from each photoacoustic ion indicator in response to the light signal; determining, based on the photoacoustic signals, a quantity of the one or more photoacoustic ion indicators exhibiting the shift; and calculating a membrane potential of the neuron based on the quantity of the one or more ion indicators exhibiting the shift.

### BRIEF DESCRIPTION OF THE DRAWINGS

The accompanying drawings, which are incorporated in and form a part of the specification, illustrate the embodiments of the technology and together with the written description serve to explain the principles, characteristics, and features of the technology. In the drawings:

FIG. 1 depicts a block diagram of an illustrative system for measuring membrane potential of a neuron in accordance with an embodiment.

FIGS. 2A-2B depict exemplary embodiments of photoacoustic probes in accordance with some embodiments.

FIG. 3 depicts a flow diagram of an illustrative method of measuring the membrane potential of a neuron in accordance with an embodiment.

FIG. 4 illustrates a block diagram of an illustrative data processing system in which embodiments may be implemented.

### DETAILED DESCRIPTION

This disclosure is not limited to the particular systems, devices and methods described, as these may vary. The terminology used in the description is for the purpose of describing the particular versions or embodiments only, and is not intended to limit the scope. Such aspects of the disclosure be embodied in many different forms; rather, these embodiments are provided so that this disclosure will be thorough and complete, and will fully convey its scope to those skilled in the art.

As used in this document, the singular forms “a,” “an,” and “the” include plural references unless the context clearly dictates otherwise. With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

As will be understood by one skilled in the art, for any and all purposes, such as in terms of providing a written description, all ranges disclosed herein are intended as encompassing each intervening value between the upper and lower limit of that range and any other stated or intervening value in that stated range. All ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells as well as the range of values greater than or equal to 1 cell and less than or equal to 3 cells. Similarly, a group having 1-5 cells refers to groups having 1, 2, 3, 4, or 5 cells, as well as the range of values greater than or equal to 1 cell and less than or equal to 5 cells, and so forth.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, structures, or groups thereof, or any members of a claimed group, less than the full measure of this disclosure can be claimed for any reason.

All percentages, parts and ratios are based upon the total weight of the compositions and all measurements made are at about 25° C., unless otherwise specified.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art. Where the context of the disclosure indicates otherwise, or is inconsistent with such an interpretation, the above-stated interpretation may be modified as would be readily apparent to a person skilled in the art. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). Further, the transitional term “comprising,” which is synonymous with “including,” “containing,” or “characterized by,” is inclusive or open-ended and does not exclude additional, unrecited elements or method steps. While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The terms “patient” and “subject” are interchangeable and may be taken to mean any living organism which contains neural tissue. As such, the terms “patient” and “subject” may include, but is not limited to, any non-human mammal, primate or human. A subject can be a mammal such as a primate, for example, a human. The term “subject” includes domesticated animals such as cats, dogs, etc., livestock (e.g., cattle, horses, swine, sheep, goats, etc.), and laboratory animals (e.g., mice, rabbits, rats, gerbils, guinea pigs, possums, etc.). In some embodiments, the patient or subject is an adult, child or infant. In some embodiments, the patient or subject is a human.

The term “tissue” refers to any aggregation of similarly specialized cells which are united in the performance of a particular function.

The term “disorder” is used in this disclosure to mean, and is used interchangeably with, the terms disease, condition, or illness, unless otherwise indicated.

The term “real-time” is used to refer to calculations or operations performed on-the-fly as events occur or input is received by the operable system. However, the use of the term “real-time” is not intended to preclude operations that cause some latency between input and response, so long as the latency is an unintended consequence induced by the performance characteristics of the machine.

Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

Throughout this disclosure, various patents, patent applications and publications are referenced. The disclosures of these patents, patent applications and publications are incorporated into this disclosure by reference in their entireties in order to more fully describe the state of the art as known to those skilled therein as of the date of this disclosure. This disclosure will govern in the instance that there is any inconsistency between the patents, patent applications and publications cited and this disclosure.

### Photoacoustic Ion Indicators

As discussed herein, it may be desirable to detect and measure changes in membrane potential of a neuron in order to track action potentials and assess neurological activity. Evaluating neurological activity provides important information that may enhance understanding of physiological mechanisms, functional behaviors of animals, various disease states, and disease etiology. As generally described herein, membrane potential may be detected by tracking ion flux with an ion indicator that selectively binds an ion of interest, such as sodium, calcium, and/or potassium. Ideally, the ion flux may be tracked in real time or close to real time, with a high temporal resolution, and with high signal-to-noise ratio. Moreover, it would be useful to track ion flux at greater depth in order to examine neurological activity of neural networks in deeper regions of living tissue (e.g., the brain).

Accordingly, embodiments of the present subject matter are directed to a photoacoustic ion indicator (PAII) for tracking ionic flux. The photoacoustic ion indicator may be a linked molecule comprising a metal chelating agent (i.e., an ionophore) configured to selectively bind to an ion involved in the neuron action potential mechanism and a highly absorbent molecule (i.e., a chromophore). These components are described in greater detail herein.

The metal chelating agent may be provided in several forms. For example, metal chelating agent may be configured to selectively bind an ion selected from sodium, potassium, and calcium. However, metal chelating agents that selectively bind other ions may also be utilized. The action potential mechanism directly involves influx or outflux of sodium, potassium, and calcium from the neuron at different stages. As such, changes in concentration of one or more of these ions within the cell is indicative of progression of the neuron through stages of the action potential mechanism. In some embodiments, the metal chelating agent selectively binds one of sodium ions, potassium ions, and calcium ions with a high degree of specificity. For example, the metal chelating agent's dissociation constant for the selected ion may be about 5 mM to about 50 mM, about 5 mM to about 25 mM, about 5 mM to about 20 mM, about 5 mM to about 15 mM, about 5 mM to about 10 mM, about 1 mM to about 5 mM, lower than about 1 mM, or individual values or ranges therebetween, indicating strong binding between the metal chelating agent and the selected ion. Further, the metal chelating agent is highly selective to the selected ion over other ions that may be present in the intracellular environment and/or other ions to which the metal chelating agent may be exposed (i.e., interfering ions). For example, the metal chelating agent's dissociation constant for the interfering ions may be about 100 mM to about 150 mM, about 125 mM to about 150 mM, about 150 mM to about 175 mM, about 175 mM to about 200 mM, greater than about 200 mM, or individual values or ranges therebetween, indicating poor binding between the metal chelating agent and the interfering ions. As a result, the quantity of metal chelating agent bound to the selected ion in the cell represents a high percentage of total bound metal chelating agent. For example, the percentage may be about 50%, about 60%, about 70%, about 80%, about 90%, about 100%, or individual values therebetween or ranges therebetween. In some alternative embodiments, the metal chelating agent may selectively bind two or more of sodium ions, potassium ions, and calcium ions. For example, the metal chelating agent may selectively bind all of sodium ions, potassium ions, and calcium ions. As such, the ion indicator may be used to track overall membrane potential through the action potential mechanism (i.e., changes in concentration of all positively charged ions involved in the action potential mechanism).

In one example, the metal chelating agent comprises 15-crown-5 ether, which selectively binds to sodium ions. Accordingly, the bound PAII would indicate sodium concentration in the cell. In some embodiments, 15-crown-5 may additionally bind potassium ions or other alkali metal ions in some quantity. However, 15-crown-5 has a higher selectivity for sodium ions. 15-crown-5 has the following basic structure:

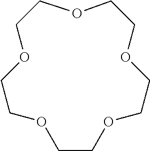 [SMILES: C1COCCOCCOCCOCCO1]

In additional embodiments, the metal chelating agent comprises an analog of 15-crown-5. For example, the metal chelating agent may comprise an aza-crown such as 1-Aza-15-crown-5, where one of the oxygen atoms is replaced by a nitrogen atom. In yet additional embodiments, the metal chelating agent comprises another analog of 15-crown-5 wherein a plurality of oxygen atoms or all of the oxygen atoms are replaced by nitrogen atoms. 1-Aza-15-crown-5 has the following basic structure:

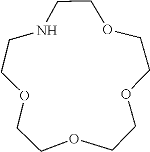 [SMILES: C1COCCOCCOCCOCCN1]

In another example, the metal chelating agent comprises 18-crown-6 ether, which selectively binds to potassium ions. Accordingly, the bound PAII would indicate potassium concentration in the cell. In some embodiments, 18-crown-6 may additionally bind sodium ions or other alkali metal ions in some quantity. However, 18-crown-6 has a higher selectivity for potassium ions. 18-crown-6 has the following basic structure:

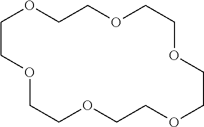 [SMILES: C1COCCOCCOCCOCCOCCO1]

In additional embodiments, the metal chelating agent comprises an analog of 18-crown-6. For example, the metal chelating agent may comprise an aza-crown such as 1-Aza-18-crown-6, where one of the oxygen atoms is replaced by a nitrogen atom. In yet additional embodiments, the metal chelating agent comprises another analog of 18-crown-6 wherein a plurality of oxygen atoms or all of the oxygen atoms are replaced by nitrogen atoms. 1-Aza-18-crown-6 has the following basic structure:

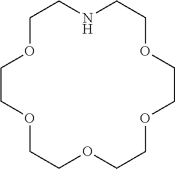 [SMILES: C1COCCOCCOCCOCCOCCN1]

In another example, the metal chelating agent comprises a BAPTA motif, which selective binds calcium ions. Accordingly, the bound PAII would indicate calcium concentration in the cell. Due to the presence of four carboxylic acid functional groups, BAPTA may bind two calcium ions. In some embodiments, however, BAPTA may be modified or configured to bind a single calcium ion. In additional embodiments, the PAII may be configured to indicate the binding of one or two calcium ions by a different absorption shift. BAPTA has the following basic structure:

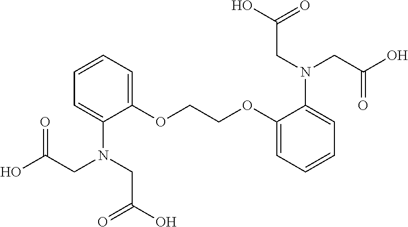 [SMILES: O=C(O)CN(CC(=O)O)c1ccccc1OCCOc1ccccc1N(CC(=O)O)CC(=O)O]

Referring again to the overall structure of the PAII, the chromophore may be provided in several forms. Because all molecules absorb light to some degree, a wide variety of candidates are available as chromophores. As the measurement of ion concentration is based on absorbance rather than fluorescence, it is not important that the chromophore exhibits fluorescence when the PAII is bound to an ion. In some embodiments, the chromophore exhibits a low amount of fluorescence. In some embodiments, the chromophore exhibits no fluorescence. The chromophore may exhibit a high amount of light absorption at a particular wavelength or range of wavelengths. The chromophore may exhibit a substantial absorption shift upon binding of the PAII. For example, the chromophore exhibits a first absorption profile when the metal chelating agent of the PAII is not bound to an ion and a second absorption profile when the metal chelating agent of the PAII is bound to an ion. Accordingly, detection of the absorption shift is indicative of binding and thus the presence of the ion. For example, a system as further described herein may use a device that is tuned or sensitive to the second absorption profile so as to quantify the bound PAII.

The absorption profiles may include a variety of characteristics. In some embodiments, the absorption profile includes an absorption wavelength, an absorption wavelength range, and/or a peak absorption wavelength. In some embodiments, the absorption profile includes an amount of absorption. In some embodiments, the absorption profile includes an absorption coefficient (μ_{α}). Accordingly, the absorption shift may comprise a shift one of the characteristics of the absorption profile (e.g., peak absorption wavelength) or a plurality of the characteristics. By changing the absorption profile, the resulting photoacoustic effect is altered in a detectable manner.

The chromophore may have a high extinction coefficient. For example the extinction coefficient may be about 10² to about 10³ M⁻¹ cm⁻¹, about 10³ to about 10⁴ M⁻¹ cm⁻¹, greater than about 10⁴ M⁻¹ cm⁻¹, or individual values or ranges therebetween. Further, the chromophore may additionally have a peak absorption wavelength of about 300 nm to about 350 nm, about 350 nm to about 400 nm, about 400 nm to about 450 nm, greater than about 450 nm, or individual values or ranges therebetween.

In some embodiments the chromophore comprises a linear acene. In some embodiments, the chromophore comprises tetracene. In some embodiments, the chromophore comprises pentacene. In additional embodiments, the chromophore comprises another linear acene such as anthracene, hexacene, or a larger acene. Alternative types of chromophore molecules may be utilized as would be apparent to one having an ordinary level of skill in the art. A wide variety of chromophore molecules may be utilized because virtually all molecules exhibit at least some light absorbance.

The metal chelating agent and the chromophore may be combined or linked in several manners as would be known to a person having an ordinary level of skill in the art. In some embodiments, the metal chelating agent and the chromophore are linked directly. In some embodiments, the metal chelating agent and the chromophore are linked through an intermediate linker molecule. For example, the metal chelating agent and the chromophore may be directly linked by N-alkylation with the chromophore in THF in the presence of triethylamine. An exemplary synthesis reaction involving 1-aza-15-crown-5 and tetracene is demonstrated below:

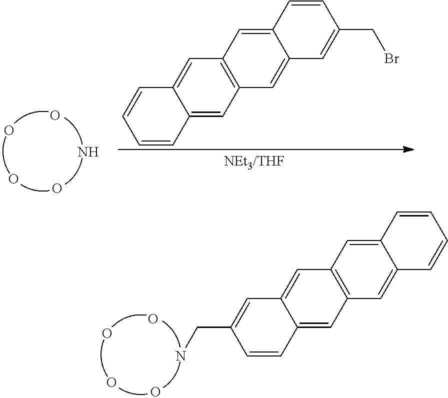

A manner of performing this reaction is described by Yoshio Nakahara et al. in “Fluorometric Sensing of Alkali Metal and Alkaline Earth Metal Cations by Novel Photosensitive Monoazacryptand Derivatives in Aqueous Micellar Solutions,” Organic & Biomolecular Chemistry 3.9 (2005): 1787-1794, which is incorporated by reference herein in its entirety. However, the reaction used to link the metal chelating agent and the chromophore may vary based on the selected metal chelating agent and the selected chromophore.

In some embodiments, a plurality of different PAIIs may be used in the manner described herein. For example, two or more PAIIs may be used where each PAII binds selectively to a different ion of sodium, potassium and calcium based on the respective metal chelating agent. Additionally, each PAII may include a chromophore having a different discernable absorption shift. Accordingly, a system as further described herein may be configured to detect each absorption shift separately and thus quantify changes in concentration of each ion in the cell. As such, this mix of PAIIs facilitates more specific and accurate tracking by the movement of each of the ions. For example, in addition to changes in overall membrane potential, information about the movement of each of sodium, potassium, and calcium at different stages may elucidate information about the mechanism and/or behavior in different disease states.

In some embodiments, the PAII comprises a single component that serves as the metal chelating agent and the chromophore. A metal chelating agent may also be a chromophore that undergoes a measurable shift in absorbance such that it performs both functions of the PAII (i.e., binding to the ion and undergoing an absorption shift). For example, in some embodiments, the PAII comprises BAPTA, which selectively binds potassium ions and undergoes a measurable absorption shift.

In additional embodiments, where the metal chelating agent is also a chromophore that undergoes a measurable shift in absorbance, the metal chelating agent may nonetheless be paired with a separate chromophore as described herein. For example, BAPTA may be paired with a chromophore as described herein such that the resulting PAII comprises two components that undergo a measurable absorption shift. Accordingly, the absorption shift of one or both of the metal chelating agent and the separate chromophore may be measured and used, individually or in combination, to assess the membrane potential of the neuronal cell. In some embodiments, the metal chelating agent and the separate chromophore may be selected to amplify a single measurable shift. For example, where both the metal chelating agent and the separate chromophore have substantially the same peak absorption wavelength after binding of the selected ion, the measurable shift at the peak absorption wavelength may be amplified due to absorbance by both the metal chelating agent and the separate chromophore, thereby providing a larger signal for detection. In another example, where both the metal chelating agent and the separate chromophore have substantially the same peak absorption wavelength prior to binding of the selected ion, the measurable negative shift at the peak absorption wavelength may be amplified due to absorbance by both the metal chelating agent and the separate chromophore, thereby providing a greater magnitude of change of signal. However, the metal chelating agent and the separate chromophore may be aligned in one or more other absorption characteristics as described herein in order to amplify the effect for detection.

The PAIIs may be loaded into the neuron cell in a variety of manner. In some embodiments, the PAIIs are applied to the tissue and actively loaded by whole-cell patch clamp electrophysiology. In some embodiments, the PAIIs are inserted within a pipette that is invasively inserted into the tissue to inject the PAIIs therein. However, in some embodiments, the PAIIs are applied to the tissue and passively loaded. In many cases, the structure of the metal chelating agents includes negatively charged polar groups that are cell-impermeable and preclude passive loading of the PAIIs across the membrane. In such cases, the metal chelating agent may be modified by polar masking. In polar masking, acetoxymethyl esters (i.e., AM esters) may be applied to the metal chelating agent to modify the negatively charged carboxylic groups and produce an uncharged chelating agent that is cell-permeable. Accordingly, the PAIIs may be passively loaded into the neuron cell. Further, once inside, the AM esters are cleaved by non-specific intracellular esterases located within the cells (i.e., as part of a natural mechanism) to return the PAII to the cell-impermeable state. Accordingly, the PAIIs are retained within the cell to a high degree.

### System for Measuring Neuronal Membrane Potential

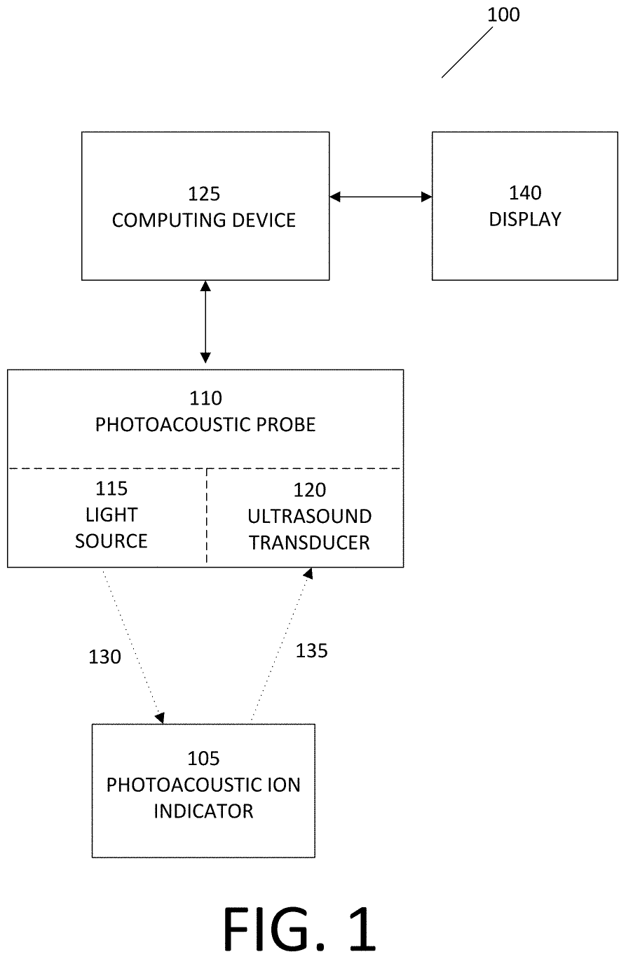

Referring now to FIG. 1, a block diagram of a system for measuring the membrane potential of a neuron is depicted in accordance with an embodiment. As shown, the system 100 comprises a photoacoustic ion indicator 105 as described herein for tracking ionic flux. The photoacoustic ion indicator 105 may be a linked molecule comprising a metal chelating agent configured to selectively bind to an ion involved in the neuron action potential mechanism and a chromophore. The system 100 further comprises a photoacoustic probe 110 comprising a light source 115 configured to emit a light signal 130 and an ultrasound transducer 120 configured to receive a photoacoustic signal 135 in response to the emitted light signal. The system 100 further comprises a computing device 125 configured to receive the photoacoustic signal 135 from the ultrasound transducer 120 and calculate, based on the photoacoustic signal 135, a membrane potential of the neuron. In some embodiments, the system may further comprise a display 140 configured to receive the membrane potential from the computing device 125 and display the membrane potential to a user.

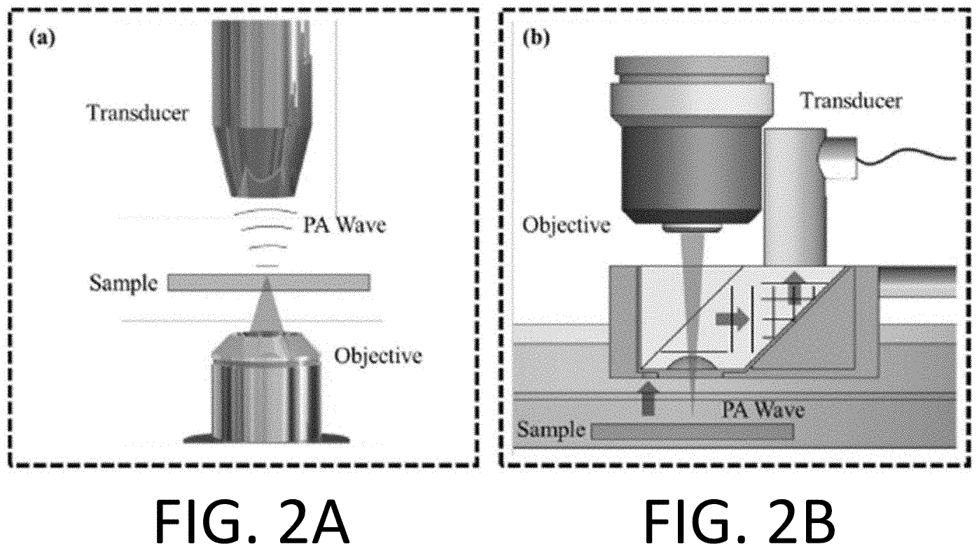

The photoacoustic probe 110 may be provided in a variety of forms. In some embodiments, the photoacoustic probe uses a high-frequency ultrasound transducer. In some embodiments, the photoacoustic probe 110 may be a photoacoustic microscopy device. For example, the photoacoustic probe 110 may be an optical resolution photoacoustic microscopy (OR-PAM) device. For example, the photoacoustic probe may be configured to detect changes in refractive index in the tissue may be used to sense the photoacoustic signal 135 because the photoacoustic signal 135 results in a change in refractive index of the material that it propagates through. In some embodiments, differential interference contrast microscopy and/or Brillouin microscopy is used. An exemplary OR-PAM arrangement is depicted in FIG. 2A. However, in some embodiments, the photoacoustic probe 110 may be an acoustic resolution photoacoustic microscopy (AR-PAM) device. The photoacoustic probe is designed to emit the light signal and receive the photoacoustic signal from the same side. An exemplary AR-PAM arrangement is depicted in FIG. 2B.

In some embodiments, the light source 115 is a laser. For example, the light source 115 may be a high-intensity laser, e.g., a nanosecond pulsed laser beam. The laser may be configured to provide fast excitation and resultant photoacoustic signal. For example, the laser may be a Bessel beam laser. However, the light source 115 may also be provided in a variety of additional forms as would be understood to a person having an ordinary level of skill in the art. In some embodiments, the photoacoustic probe 110 may further comprise a reflective surface (e.g., a mirror) to direct the light signal away from the photoacoustic probe 110 (e.g., through an aperture) and towards the tissue. In some embodiments, the reflective surface may be movable to adjust the direction of the light signal.

In some embodiments, the photoacoustic probe includes additional components. In some embodiments, the photoacoustic probe includes an ultrasound transmission line, a light transmission line, an ultrasound receiver, and/or an amplifier. In some embodiments, the photoacoustic probe includes a plurality of a described component. For example, the photoacoustic probe may include a plurality of ultrasound transducers and/or lasers.

The computing device 125 is configured to receive the photoacoustic signal 135 and calculate the membrane potential. This is performed based on the known and understood principles of the photoacoustic effect. In essence, the emitted light signal creates a resultant sound signal. When molecules (i.e., the chromophores of the PAIIs) absorb light at specific wavelengths, the result is molecular excitation and thermal expansion of the tissue that generates an acoustic wave. The computing device 125 may receive several parameters through input and/or calibration in order to calculate the membrane potential. Particularly, the computing device 125 may have information related to the absorption shift of the chromophore. As such, the computing device (or alternatively, the photoacoustic probe) is tuned to identify the second absorption profile (i.e., absorption profile of the chromophore in the bound state as further described herein) based on the known absorption shift. For example, the initial pressure wave (P₀) of the photoacoustic signal may be expressed as:

P₀=Γμ_{α}(λ)F

where Γ is the Gruneisen parameter, μ_{α} is the absorption coefficient for a particular wavelength λ of emitted light, and F is the fluence. Shifts in μ_{α} (i.e., the absorption shift) results in changes to the pressure wave (i.e., the photoacoustic signal). By measuring the changes to the photoacoustic signal, a quantity of the PAII that has undergone the absorption shift may be calculated, which is indicative of fluctuation in the ion concentration and thus membrane potential. A larger quantity of PAII exhibiting the second absorption profile is indicative of a larger concentration of ions in the cell. Similarly, a larger change in the overall photoacoustic signal is indicative of a larger fluctuation in ion concentration.

In some embodiments, the computing device 125 and/or the display 140 may be used to record and monitor membrane potential in real time. For example, the system 100 may be used to repeatedly collect measurements over a period of time and may be displayed and updated in real time. In some embodiments, a stimulus may be applied to the tissue or another test may be performed during collection of measurements in order to record a response. In some embodiments, a drug, a biologic, or a chemopharmaceutical may be applied to the tissue in order to record an effect of the drug, biologic, or chemopharmaceutical on the behavior of the neurons (e.g., firing patterns).

### Method of Measuring Neuronal Membrane Potential

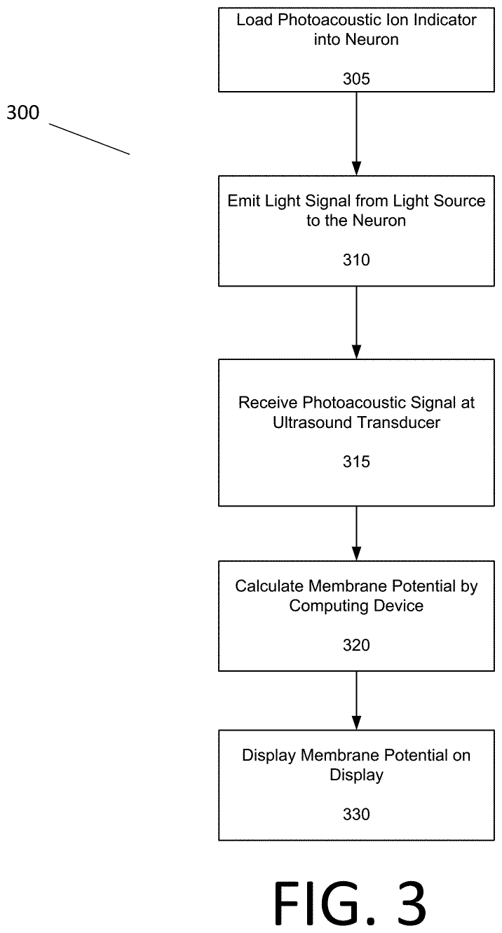

Referring now to FIG. 3, a flow diagram of an illustrative method of measuring the membrane potential of a neuron is depicted in accordance with an embodiment. As shown, the method 300 comprises loading 305 a quantity of photoacoustic ion indicator into the neuron, the photoacoustic ion indicator comprising a metal chelating agent configured to selectively bind to an ion involved in the neuron action potential mechanism and a chromophore molecule. The PAII may comprise any of the embodiments and/or characteristics as described herein. The method further comprises emitting 310 a light signal to the neuron by a light source of the photoacoustic probe and receiving 315 a photoacoustic signal by an ultrasound transducer of the photoacoustic probe in response to the light signal. The method further comprises receiving the photoacoustic signal by a computing device and calculating 320 the membrane potential of the neuron based on the photoacoustic signal.

In some embodiments, the method 300 comprises calculating the membrane potential of a single cell. In some embodiments, the method 300 comprises calculating the membrane potential of a plurality of cells. For example, a plurality of simultaneously firing neurons may be tracked by the method 300 described herein. In some embodiments, the method 300 comprises monitoring the membrane potential of one or more cells over a period of time. For example, the method 300 may be repeated several times over a short duration in order to track the behavior of the cells through the stages of the action potential mechanism.

In some embodiments, the membrane potential is calculated 320 by determining a quantity of the photoacoustic ion indicator that is exhibiting an absorption shift indicative of the binding of an ion (i.e., one or sodium ions, potassium ions, and calcium ions). For example, the absorption shift may be a shift in absorption wavelength range, peak absorption wavelength, a total absorption value, and/or an absorption coefficient. The absorption shift may have any of the characteristics as described herein with respect to the PAII and/or the system 100. The absorption shift may result in a detectable change in the photoacoustic signal received 315 by the ultrasound transducer. Accordingly, the computing device may use the photoacoustic signal to determine a quantity of the photoacoustic ion indicator that is exhibiting the shift based on the predetermined or expected absorption shift of the photoacoustic ion indicator upon binding and the predetermined or expected effect of the absorption shift on the photoacoustic signal. For example, the computing device may use one or more equations or known relationships between the absorption shift and the photoacoustic signal to determine the quantity of the photoacoustic ion indicators exhibiting the absorption shift. Based on the determined quantity, the computing device may calculate 320 the membrane potential of the neuron.

In some embodiments, the method 300 further comprising displaying 330 the calculated membrane potential on a display connected to the computing device. In some embodiments, additional information may be displayed on the computing device. For example, where a plurality of measurements have been collected, the measurements may be displayed in an aggregate form, such as a chart, graphic, table, profile, or other format.

In some embodiments, the computing device and/or the display may be used to record and monitor membrane potential in real time. For example, the system may be used to repeatedly collect measurements over a period of time and may be displayed and updated in real time. In some embodiments, a stimulus may be applied to the tissue or another test may be performed during collection of measurements in order to record a response. In some embodiments, a drug, a biologic, or a chemopharmaceutical may be applied to the tissue in order to record an effect of the drug, biologic, or chemopharmaceutical on the behavior of the neurons (e.g., firing patterns).

In some embodiments, the method 300 is used in vitro for research or testing purposes. However, in additional embodiments, the method 300 may be used in vivo to record the behavior of live tissue. For example, the method 300 may be used on a subject such a mouse, a human, or other laboratory animals. The method 300 may be used to study neural mechanisms, neural diseases and disorders, and/or to functional behavior of animals. In some embodiments, the method 300 may be used in clinical settings, for example for diagnosing conditions in a subject and/or evaluating a subject's behavior.

While the described embodiments are discussed with respect to tracking the action potential in neurons, the apparatuses, systems, and methods described herein may be adapted for other types of cells that exhibit a fluctuating membrane potential as would be apparent to a person having an ordinary level of skill in the art. For example, the apparatuses, systems, and methods may be adapted to track the action potential of cardiomyocytes to evaluate cardiovascular activity and health.

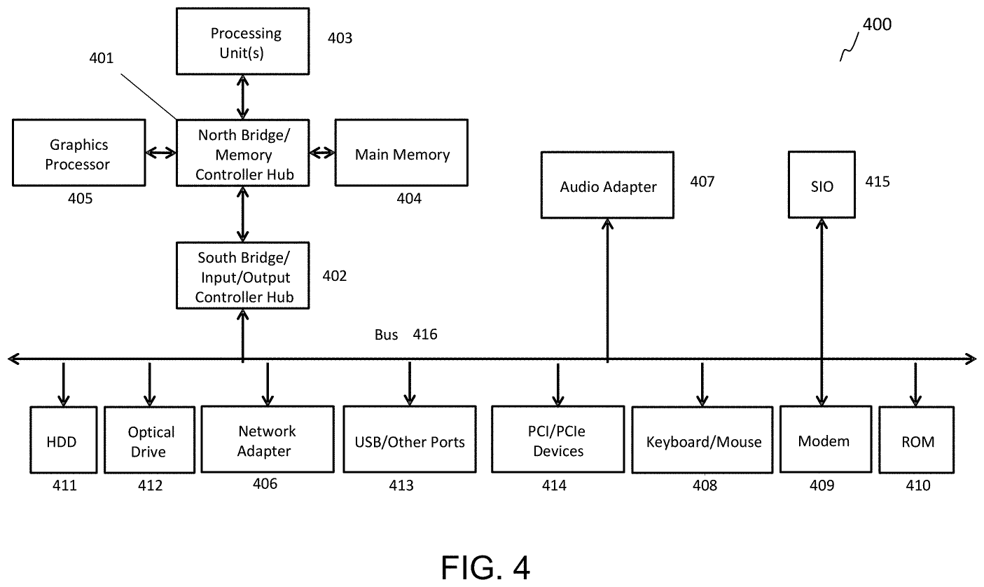

FIG. 4 illustrates a block diagram of an illustrative data processing system 400 in which aspects of the illustrative embodiments are implemented. The data processing system 400 is an example of a computer, such as a server or client, in which computer usable code or instructions implementing the process for illustrative embodiments of the present technology are located. In some embodiments, the data processing system 400 may be a server computing device. For example, data processing system 400 can be implemented in a server or another similar computing device. The data processing system 400 can be configured to, for example, transmit and receive information related to the light signal, photoacoustic signal and/or membrane potential.

In the depicted example, data processing system 400 can employ a hub architecture including a north bridge and memory controller hub (NB/MCH) 401 and south bridge and input/output (I/O) controller hub (SB/ICH) 402. Processing unit 403, main memory 404, and graphics processor 405 can be connected to the NB/MCH 401. Graphics processor 405 can be connected to the NB/MCH 401 through, for example, an accelerated graphics port (AGP).

In the depicted example, a network adapter 406 connects to the SB/ICH 402. An audio adapter 407, keyboard and mouse adapter 408, modem 409, read only memory (ROM) 410, hard disk drive (HDD) 411, optical drive (e.g., CD or DVD) 412, universal serial bus (USB) ports and other communication ports 413, and PCI/PCIe devices 414 may connect to the SB/ICH 402 through bus system 416. PCI/PCIe devices 414 may include Ethernet adapters, add-in cards, and PC cards for notebook computers. ROM 410 may be, for example, a flash basic input/output system (BIOS). The HDD 411 and optical drive 412 can use an integrated drive electronics (IDE) or serial advanced technology attachment (SATA) interface. A super I/O (SIO) device 415 can be connected to the SB/ICH 402.

An operating system can run on the processing unit 403. The operating system can coordinate and provide control of various components within the data processing system 400. As a client, the operating system can be a commercially available operating system. An object-oriented programming system, such as the Java' programming system, may run in conjunction with the operating system and provide calls to the operating system from the object-oriented programs or applications executing on the data processing system 400. As a server, the data processing system 400 can be an IBM® eServer™ System® running the Advanced Interactive Executive operating system or the Linux operating system. The data processing system 400 can be a symmetric multiprocessor (SMP) system that can include a plurality of processors in the processing unit 403. Alternatively, a single processor system may be employed.

Instructions for the operating system, the object-oriented programming system, and applications or programs are located on storage devices, such as the HDD 411, and are loaded into the main memory 404 for execution by the processing unit 403. The processes for embodiments described herein can be performed by the processing unit 403 using computer usable program code, which can be located in a memory such as, for example, main memory 404, ROM 410, or in one or more peripheral devices.

A bus system 416 can be comprised of one or more busses. The bus system 416 can be implemented using any type of communication fabric or architecture that can provide for a transfer of data between different components or devices attached to the fabric or architecture. A communication unit such as the modem 409 or the network adapter 406 can include one or more devices that can be used to transmit and receive data.

Those of ordinary skill in the art will appreciate that the hardware depicted in FIG. 4 may vary depending on the implementation. Other internal hardware or peripheral devices, such as flash memory, equivalent non-volatile memory, or optical disk drives may be used in addition to or in place of the hardware depicted. Moreover, the data processing system 400 can take the form of any of a number of different data processing systems, including but not limited to, client computing devices, server computing devices, tablet computers, laptop computers, telephone or other communication devices, personal digital assistants, and the like. Essentially, data processing system 400 can be any known or later developed data processing system without architectural limitation.

Although the present invention has been described in considerable detail with reference to certain preferred embodiments thereof, other versions are possible. Therefore the spirit and scope of the appended claims should not be limited to the description and the preferred versions contained within this specification. Various aspects of the present invention will be illustrated with reference to the following non-limiting examples:

### EXAMPLES

### Example 1

### Development of PAM Systems to Measure Absorption Shifts in PAIIs

Photoacoustic microscopy (PAM) systems typically involve raster scanning optical and acoustic focal points, which are confocally aligned. Systems are categorized as optical resolution (OR-PAM) or acoustic resolution (AR-PAM) as determined by the sharper focal point. These systems will be investigated for in vitro and in vivo applications.

### Example 1.1

### Investigation of Photoacoustic Wave Generation and Detection

Methods. The frame rate required to visualize multiple simultaneously firing neurons will provide major challenges to most reported PAM systems, emphasizing a need for faster excitation and detection. Compared to point scanning or full sample illumination, Bessel beams may provide a successful method for fast signal generation over a 3D volume while still maintaining high resolution. Easily replicable light sheet microscopy systems retrofitted with high frequency transducer arrays in combination with existing time reversal algorithms may allow for the development of high-speed PAM systems capable of detecting changes in membrane potential across several neurons. These methods will be tested to evaluate frame rate and determine feasibility.

Photoacoustic waves change the refractive index of the material they propagate through. Two different optical techniques may be capable of detecting these changes in the refractive index including differential interference contrast (DIC) microscopy and Brillouin microscopy. These optical techniques will be tested to evaluate sensitivity to refractive index and determine feasibility.

Anticipated Results. Testing of the potential methods and techniques described will yield a PAM system with the requisite frame rate and sensitivity to detect fluctuations in membrane potential.

### Example 1.2

### Development of OR-PAM System for Monitoring Action Potentials

Methods. An OR-PAM system will be designed and assessed in terms of frame rate to determine whether it is capable of monitoring single cells in culture (i.e., in vitro imaging). The system may be designed with a light source and a transducer on opposing sides of the culture sample. A potential OR-PAM system configuration is illustrated in FIG. 2A.

Anticipated Results. Development and assessment of OR-PAM systems will yield an OR-PAM system with the requisite resolution to monitor single cells in in vitro cultures and the requisite sensitivity to detect fluctuations in membrane potential.

### Example 1.3

### Development of AR-PAM System for Monitoring Action Potentials

Methods. An AR-PAM system will be designed and assessed in terms of frame rate to determine whether it is capable of visualizing multiple simultaneously firing neurons in in vivo environments as well as in vitro environments. The system must be capable of confocal optical and acoustic alignment from the same side of the tissue or sample. A potential AR-PAM system configuration is illustrated in FIG. 2B.

Anticipated Results. Development and assessment of OR-PAM systems will yield an OR-PAM system with the requisite resolution to monitor neurons in vivo and the requisite sensitivity to detect fluctuations in membrane potential.

### Example 2

### Development of PAIIs for Na⁺, K⁺, and Ca²⁺ Ions

Absorption-based PAIIs will be synthesized to determine ion concentration in cells based on the photoacoustic effect. Several metal chelating agents and chromophores will be synthesized for each of Na+, K+, and Ca2+ ions and assessed for several properties to determine feasibility.

### Example 2.1

### Synthesis of PAIIs

Methods. The 15-crown-5 ether, 18-crown-6 ether, and BAPTA will be utilized as motifs for selectively binding Na⁺, K⁺, and Ca²⁺, respectively. Synthesis of ionophores and ion indicators derived from crown ether motifs will be used to guide the development of PAIIs. Synthesis may involve simple one step reactions utilizing commercially available reagents to bind highly conjugated chromophores to the crown ether motif. A similar approach will be utilized for the BAPTA motif, which itself already undergoes a measurable shift in absorbance without additional chromophores. Successful synthesis will be determined using IR, NMR, and mass spectroscopy.

Anticipated Results. Simple one step reactions between ionophores and ion indicators as described will yield stable, linked molecules.

### Example 2.2

### Screening of PAIIs for Selectivity and Absorption Spectrum Shifts

Methods. Synthesized PAIIs will be screened with the following criteria: (1) dissociation constant k_{d}, of 5-50 mM for the ion of interest; (2) selective over interfering ions, e.g., k_{d}>150 mM for interfering ions; (3) extinction coefficient >10³ M⁻¹ cm⁻¹; (4) peak absorption >350 nm; (5) undergo large μ_{α} shift after binding (e.g., a shift of about 50 nm, about 100 nm, about 200 nm, greater than about 200 nm, or individual values or ranges therebetween); and (6) sufficient polar groups. Synthesized PAIIs will be compared to commercially available fluorescent ion indicators for Na⁺, K⁺, and Ca²⁺ ions utilizing UV-VIS spectroscopy. Peak absorption wavelengths of PAIIs will be used to monitor change in photoacoustic signal.

Anticipated Results. Screening of the PAIIs as described will yield one or more PAIIs meeting all criteria for each of Na⁺, K⁺, and Ca²⁺ ions. The PAIIs will demonstrate an ability to produce signals that monitor change in photoacoustic signal in a manner comparable or greater than commercially available fluorescent ion indicators.

### Example 2.3

### Using PAIIs for Measuring Changes in Membrane Potential

Methods. PAIIs can be loaded into cells through whole-cell patch clamp electrophysiology or passive cell loading by masking polar groups with acetoxymethyl esters. The ability of PAIIs to relate changes in membrane potential will be compared to commercially available fluorescent ion indicators for Na⁺, K⁺, and Ca²⁺ ions.

Anticipated Results. Testing of the PAIIs for measuring changes in membrane potential will reveal that PAIIs meeting all criteria are viable tools for monitoring ion concentration changes with comparable or better accuracy as commercially available fluorescent ion indicators.

### Example 3

### Synthesis Reactions for PAIIs

### Example 3.1

### Synthesis of PAII Including 1-aza-15-crown-5 and Tetracene

Methods. 1-aza-15-crown-5, which is a metal chelating agent selective to Na⁺ ions, may be bound to tetracene, which is a chromophore, by the following reaction:

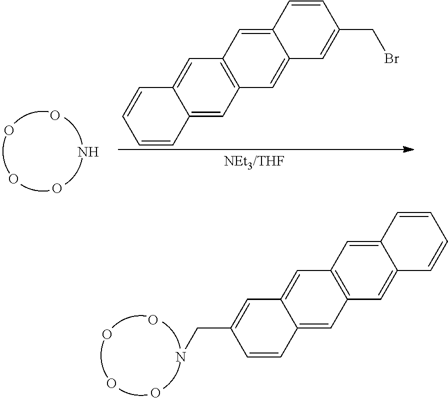

Anticipated Results. Synthesis of 1-aza-15-crown-5 to tetracene by the described reaction will yield a photoacoustic ion indicator exhibiting selectivity to Na⁺ ions and exhibiting an absorption shift upon binding of Na⁺ ions.

### Example 3.2

### Synthesis of PAII Including 1-aza-15-crown-5 and Pentacene

Methods. 1-aza-15-crown-5, which is a metal chelating agent selective to Na+ ions, may be bound to pentacene, which is a chromophore, by the following reaction:

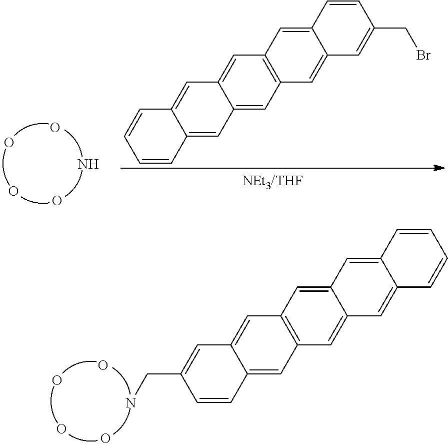

Anticipated Results. Synthesis of 1-aza-15-crown-5 to pentacene by the described reaction will yield a photoacoustic ion indicator exhibiting selectivity to Na⁺ ions and exhibiting an absorption shift upon binding of Na⁺ ions.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Instead, this application is intended to cover any variations, uses, or adaptations of the present teachings and use its general principles. Further, this application is intended to cover such departures from the present disclosure as come within known or customary practice in the art to which these teachings pertain. Many modifications and variations can be made to the particular embodiments described without departing from the spirit and scope of the present disclosure as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. A photoacoustic ion indicator for detecting ion concentration within a neuron, the ion indicator comprising:
a metal chelating agent comprising one or more polar groups, wherein the metal chelating agent is configured to selectively bind to an ion selected from the group consisting of sodium, calcium, and potassium;
a chromophore linked to the metal chelating agent, wherein the chromophore molecule exhibits a shift of at least one light absorption characteristic upon binding of the metal chelating agent to the ion; and
one or more acetoxymethyl esters bound to the one or more polar groups and configured to be cleaved from the one or more polar groups by an esterase within the neuron,
wherein the photoacoustic ion indicator is permeable through a membrane of the neuron when the one or more acetoxymethyl esters are bound to one or more polar groups, and
wherein the photoacoustic ion indicator is impermeable through the membrane of the neuron when the one or more acetoxymethyl esters are cleaved from the one or more polar groups.

2. The photoacoustic ion indicator of claim 1, wherein the ion is a sodium ion, and
wherein the metal chelating agent comprises 15-crown-5 ether configured to selectively bind to the sodium ion.

3. The photoacoustic ion indicator of claim 1, wherein the ion is a calcium ion, and
wherein the metal chelating agent comprises BAPTA motif configured to selectively bind to the calcium ion.

4. The photoacoustic ion indicator of claim 1, wherein the ion is a potassium ion, and
wherein the metal chelating agent comprises 18-crown-6 ether configured to selectively bind to the potassium ion.

5. The photoacoustic ion indicator of claim 1, wherein the photoacoustic ion indicator has a substantially neutral charge when the one or more acetoxymethyl esters are bound to one or more polar groups, and
wherein the photoacoustic ion indicator has a substantially negative charge when the one or more acetoxymethyl esters are cleaved from the one or more polar groups.

6. The photoacoustic ion indicator of claim 1, wherein a dissociation constant of the metal chelating agent binding the ion is less than about 50 mM.

7. The photoacoustic ion indicator of claim 1, wherein the chromophore has an extinction coefficient greater than about 103 M⁻¹ cm⁻¹.

8. The photoacoustic ion indicator of claim 1, wherein the chromophore comprises a linear acene.

9. The photoacoustic ion indicator of claim 1, wherein the at least one light absorption characteristic comprises one or more of an absorption wavelength range, a peak absorption wavelength, a total absorption value, and an absorption coefficient.

10. The photoacoustic ion indicator of claim 9, wherein the chromophore has a peak absorption wavelength greater than about 350 nm after the shift.

11. A system for measuring the membrane potential of a neuron, the system comprising:
one or more photoacoustic ion indicators, each photoacoustic ion indicator comprising:
a metal chelating agent configured to selectively bind to an ion selected from the group consisting of sodium, calcium, and potassium; and
a chromophore linked to the metal chelating agent, wherein the chromophore exhibits a shift of at least one light absorption characteristic upon binding of the metal chelating agent to the ion;
a photoacoustic probe comprising:
a laser configured to emit a light signal, wherein the chromophore is configured to absorb the light signal, and
an ultrasound transducer configured to receive a photoacoustic signal from each photoacoustic ion indicator in response to the light signal;
a processor; and
a non-transitory, computer-readable medium storing instructions that, when executed, cause the processor to:
receive the photoacoustic signals from the ultrasound transducer;
determine, based on the photoacoustic signals, a quantity of the one or more photoacoustic ion indicators exhibiting the shift; and
calculate a membrane potential of the neuron based on the quantity of the one or more ion indicators exhibiting the shift.

12. The system of claim 11, wherein the ion is a sodium ion, and
wherein the metal chelating agent comprises 15-crown-5 ether configured to selectively bind to the sodium ion.

13. The system of claim 11, wherein the ion is a calcium ion, and
wherein the metal chelating agent comprises BAPTA motif configured to selectively bind to the calcium ion.

14. The system of claim 11, wherein the ion is a potassium ion, and
wherein the metal chelating agent comprises 18-crown-6 ether configured to selectively bind to the potassium ion.

15. The system of claim 11, wherein the photoacoustic ion indicator is configured to be loaded into the neuron by whole-cell patch clamp electrophysiology.

16. The system of claim 1, wherein the photoacoustic ion indicator is configured to be loaded into the neuron by passive cell loading,

17. The system of claim 16, wherein the photoacoustic ion indicator further comprises one or more acetoxymethyl esters bound to one or more polar groups of the metal chelating agent and configured to be cleaved from the one or more polar groups by an esterase within the neuron,
wherein the photoacoustic ion indicator is permeable through a membrane of the neuron when the one or more acetoxymethyl esters are bound to one or more polar groups, and
wherein the photoacoustic ion indicator is impermeable through the membrane of the neuron when the one or more acetoxymethyl esters are cleaved from the one or more polar groups.

18. The photoacoustic ion indicator of claim 1, wherein the photoacoustic ion indicator has a substantially neutral charge when the one or more acetoxymethyl esters are bound to one or more polar groups, and
wherein the photoacoustic ion indicator has a substantially negative charge when the one or more acetoxymethyl esters are cleaved from the one or more polar groups.

19. The system of claim 1, wherein the at least one light absorption characteristic comprises one or more of an absorption wavelength range, a peak absorption wavelength, a total absorption value, and an absorption coefficient.

20. The system of claim 11, wherein the chromophore has a first absorption coefficient when the metal chelating agent is unbound and a second absorption coefficient, different from the first absorption coefficient, when the metal chelating agent is bound to the ion, and
wherein instructions that cause the processor to determine a quantity of the one or more ion indicators exhibiting the shift comprise instructions that, when executed, cause the processor to determine a quantity of the one or more photoacoustic ion indicators having the second absorption coefficient.

21. The system of claim 11, wherein the chromophore has a first peak absorption wavelength when the metal chelating agent is unbound and a second peak absorption wavelength, different from the first peak absorption wavelength, when the metal chelating agent is bound to the ion, and
wherein instructions that cause the processor to determine a quantity of the one or more ion indicators exhibiting the shift comprise instructions that, when executed, cause the processor to determine a quantity of the one or more photoacoustic ion indicators having the second peak absorption wavelength.

22. A method of measuring the membrane potential of a neuron, the method comprising:
loading one or more photoacoustic ion indicators into the neuron, wherein each photoacoustic ion indicator comprises:
a metal chelating agent configured to selectively bind to an ion selected from the group consisting of sodium, calcium, and potassium, and
a chromophore linked to the metal chelating agent, wherein the chromophore exhibits a shift of at least one light absorption characteristic upon binding of the metal chelating agent to the ion;
emitting, by a light source, a light signal configured to be absorbed by the chromophore;
receiving, by a photoacoustic probe, a photoacoustic signal from each photoacoustic ion indicator in response to the light signal;
determining, based on the photoacoustic signals, a quantity of the one or more photoacoustic ion indicators exhibiting the shift; and
calculating a membrane potential of the neuron based on the quantity of the one or more ion indicators exhibiting the shift.
